(19) United States
(12) Patent Application Publication
(10) Pub. No.: US 2006/0265076 A1
(43) Pub. Date: Nov. 23, 2006

(54) Catheter holder for spinal implant

(75) Inventors: Benjamin F. Carter, Eden Prairie, MN (US); Erik O. Martz, Savage, MN (US); Scott G. Hook, Edina, MN (US); Mark A. Rydell, Golden Valley, MN (US); Ronald Burke, Deephaven, MN (US); Robert E. Kohler, Lake Elmo, MN (US); Steven J. Healy, Vadnais Heights, MN (US); Bruce R. Bowman, Eden Prairie, MN (US); John Sherman, Wayzata, MN (US); Jean-Charles Lehuec, Bordeaux (FR)

Correspondence Address: FAEGRE & BENSON LLP;PATENT DOCKETING, 2200 WELLS FARGO CENTER, 90 SOUTH SEVENTH STREET, MINNEAPOLIS, MN, 55402, US

(73) Assignee: Disc Dynamics, Inc., Eden Prairie, MN (US)

(21) Appl. No.: 11/268,876

(22) Filed: Nov. 8, 2005

Related U.S. Application Data
(60) Provisional application No. 60/677,273, filed on May 3, 2005.
(60) Provisional application No. 60/708,245, filed on Aug. 15, 2005.
(60) Provisional application No. 60/708,244, filed on Aug. 15, 2005.

(51) Int. Cl. A61F 2/44

(52) U.S. Cl. 623/17.16; 606/108

(57) ABSTRACT

A catheter holder designed to deliver a curable biomaterial to an intervertebral disc space. By securing the catheter to the catheter holder, the depth of insertion of the catheter into the disc space can be accurately controlled. The catheter holder optionally helps with insertion of a optional mold through the annulotomy and into the disc space created by the nuclectomy. The catheter holder helps keep the mold from being drawn too far into the disc space or pushed too far out of the disc space during polymer injection.

The present application claims the benefit of U.S. Provisional Application Ser. No. 60/677,273 entitled Catheter Holder for Spinal Implants filed May 3, 2005; U.S. Provisional Application Ser. No. 60/708,245 entitled Catheter Holder for Spinal Implants filed Aug. 15, 2005; U.S. Provisional Application Ser. No. 60/708,244 entitled Multi-Lumen Mold For Intervertebral Prosthesis And Method Of Using Same filed on Aug. 15, 2005, all of which are hereby incorporated by reference.

### FIELD OF THE INVENTION

The present invention relates to a method and apparatus for filling an intervertebral disc space with an in situ curable biomaterial using a catheter holder to releasably secure the delivery mechanism to the patient.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulposus, and annulus fibrosus. The central portion, the nucleus pulposus or nucleus, is relatively soft and gelatinous; being composed of about 70 to 90% water. The nucleus pulposus has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to a number of injuries. Disc herniation occurs when the nucleus begins to extrude through an opening in the annulus, often to the extent that the herniated material impinges on nerve roots in the spine or spinal cord. The posterior and posterio-lateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine. Intervertebral disc implants for spinal fusion are known in the art as well, such as disclosed in U.S. Pat. Nos. 5,425,772 (Brantigan) and U.S. Pat. No. 4,834,757 (Brantigan).

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization.

Apparatuses and/or methods intended for use in disc repair have also been described but none appear to have been further developed, and certainly not to the point of commercialization. See, for instance, French Patent Appl. No. FR 2 639 823 (Garcia) and U.S. Pat. No. 6,187,048 (Milner et al.). Both references differ in several significant respects from each other and from the apparatus and method described below. For instance, neither reference teaches switching the flow of biomaterial between discrete operating parameters or methods of detecting ruptures in the mold. Further, neither reference teaches shunting an initial portion of a curing biomaterial in the course of delivering the biomaterial to the disc space.

Prosthetic implants formed of biomaterials that can be delivered and cured in situ, using minimally invasive techniques to form a prosthetic nucleus within an intervertebral disc have been described in U.S. Pat. No. 5,556,429 (Felt) and U.S. Pat. No. 5,888,220 (Felt et al.), and U.S. Patent Publication No. US 2003/0195628 (Felt et al.), the disclosures of which are incorporated herein by reference. The disclosed method includes, for instance, the steps of inserting a collapsed mold apparatus (which in a preferred embodiment is described as a “mold”) through an opening within the annulus, and filling the mold to the point that the mold material expands with a flowable biomaterial that is adapted to cure in situ and provide a permanent disc replacement. Related methods are disclosed in U.S. Pat. No. 6,224,630 (Bao et al.), entitled “Implantable Tissue Repair Device” and U.S. Pat. No. 6,079,868 (Rydell), entitled “Static Mixer”, the disclosures of which are incorporated herein by reference.

### BRIEF SUMMARY OF THE INVENTION

The present invention relates to a method and apparatus for filling an intervertebral disc space with an in situ curable biomaterial using a catheter holder to releasably secure the delivery mechanism to the patient. The present catheter holder can be used, for example, to implant a prosthetic total disc, or a prosthetic disc nucleus, using minimally invasive techniques that leave the surrounding disc tissue substantially intact. The phrase intervertebral disc prosthesis is used generically to refer to both of these variations.

Minimally invasive refers to a surgical mechanism, such as microsurgical, percutaneous, or endoscopic or arthroscopic surgical mechanism, that can be accomplished with minimal disruption of the pertinent musculature, for instance, without the need for open access to the tissue injury site or through minimal incisions (e.g., incisions of less than about 4 cm and preferably less than about 2 cm). Such surgical mechanism are typically accomplished by the use of visualization such as fiber optic or microscopic visualization, and provide a post-operative recovery time that is substantially less than the recovery time that accompanies the corresponding open surgical approach.

The present catheter holder is designed to position and secure the catheter in the desired position in an intervertebral disc space during and following polymer injection. The present catheter holder can be used to inject biomaterial with or without a mold.

In embodiments using a mold, the present catheter holder helps with insertion of the mold through the annulotomy and into the disc space created by the nuclectomy. By securing the catheter to the present catheter holder, the depth of insertion of the mold into the disc space can be accurately controlled during the delivery of biomaterial. In particular, the present catheter holder helps keep the mold from being drawn too far into the disc space or pushed too far out of the disc space during polymer injection. The present catheter holder can also receive other devices, such as a catheter cutter that cuts the catheter to the desired depth, imaging devices, and the like.

In one embodiment, the catheter holder includes a member having at least one catheter channel, a proximal end and a distal end. A mounting flange is attached near the proximal end of the member. In one embodiment, the mounting flange is connected, directly or indirectly, to a secondary holding device.

A surgical tool is preferably attached near the distal end of the member. In one embodiment, the surgical tool limits the depth of insertion into the intervertebral disc space. For example, the surgical tool may have a cross-sectional area greater than the cross-sectional area of the distal end of the member. The surgical tool is optionally releasably attached to the member. In one embodiment, a plurality of surgical tools having different geometric features from which the surgeon can select are provided.

In another embodiment, the surgical tool is a nerve guard ring or a blood vessel retractor. The nerve guard ring preferably has an asymmetrical support surface. The surgical tool can also serve to support the annulus during injection of the biomaterial. In one embodiment, the surgical tool is both a nerve guard ring and a support structure for the annulus. In another embodiment, the surgical tool is a support structure is delivered through the catheter to a located inside the annulus. After the biomaterial is delivered and/or at least partially cured, the support structure is removed through the catheter. Alternatively, the support structure can be detached from the member and retained in the annulus. In one embodiment, the support structure is bio-resorbable.

The member is preferably a hollow structure in fluid communication with the catheter channel. In one embodiment, the member includes an aperture in a side that is fluidly coupled to the catheter channel. The aperture is preferably configured to direct the catheter into the catheter channel at an acute angle.

A securing mechanism is provided that secures a catheter extending through the catheter channel to the catheter holder. In one embodiment, the securing mechanism is a catheter locking pin sized to fit into the catheter channel. The catheter locking pin has a distal end configured to compressively secure a catheter in the catheter channel. The catheter locking pin can optionally include a spring portion. In another embodiment, the securing mechanism is a compression fitting adapted to secure a catheter in the catheter channel.

The present invention is also directed to a catheter holder that can be secured to the patient with or without a secondary holding device. In one embodiment, an outer tube surrounds the catheter. A flexible material attaches the distal end of the catheter to the distal end of the outer tube. Displacement of the outer tube toward the distal end of the catheter causes the flexible material to expand outwardly away from the catheter. The expanded structure can be located in the annulotomy, inside the annulus, outside the annulus, or a combination thereof.

In another embodiment, the catheter holder includes an expandable bladder located near at least the distal end of the tubular member. A delivery tube is fluidly coupled to the expandable bladder. Again, the expanded bladder can be located in the annulotomy, inside the annulus, outside the annulus, or a combination thereof. In one embodiment, the expandable bladder is attached directly to a balloon catheter. In another embodiment, a sheath extends around a portion of the expandable bladder to limit the location and/or amount of expansion.

The present invention is also directed to an expandable coiled material located near the distal end of a tubular member. The proximal end of the expandable coiled material is attached to the tubular member near the distal end of the tubular member. The expandable coiled material has an outer diameter that contracts when a force directed away from the tubular member is applied to the distal end of the expandable coiled material. In operation, a tension force is applied to the coiled materials so it contracts around the catheter. The contracted coiled material is inserted into the annulotomy. The tension force is then release so the coiled material expands into the annulotomy, to secure the catheter thereto. When the procedure is competed, the tension force is again applied to the distal end of the coiled material to permit removal from the annulotomy.

The present method and apparatus include a radiopaque portion on the catheter holder to assist in imaging the position of the mold in the intervertebral disc space before, during and/or after delivery of biomaterial.

### BRIEF DESCRIPTION OF THE SEVERAL VIEWS OF THE DRAWINGS

FIG. 1 is an exemplary catheter and mold in accordance with the present invention.

FIG. 2 is a side view of a catheter holder in accordance with the present invention.

FIG. 3 is a top view of an extension on the catheter holder of FIG. 2.

FIG. 4 is an enlarged view of a cut-out in the member of FIG. 2.

FIG. 5 is a longitudinal-sectional view of an alternate cut-out in the member of FIG. 2.

FIG. 6 is an enlarged view of the member of FIG. 2.

FIG. 7 is a bottom view of the member of FIG. 6.

FIG. 8 is a top view of an alternate catheter holder in accordance with the present invention.

FIG. 9 is a side view of the catheter holder of FIG. 8.

FIGS. 10 and 11 are side views of an alternate core with a spring region for use with the catheter holder of the present invention.

FIGS. 12 and 13 are front and side views of an alternate catheter holder in accordance with the present invention.

FIG. 14 is a side view of an alternate catheter holder in accordance with the present invention.

FIG. 15 is a side view of an alternate catheter holder in accordance with the present invention.

FIG. 16 is a side view of an alternate catheter holder in accordance with the present invention.

FIGS. 17A and 18A are side views of an alternate catheter holder engaged with the annulotomy in accordance with the present invention.

FIGS. 17B and 18B are side views of an alternate catheter holder engaged above the annulotomy in accordance with the present invention.

FIGS. 19A and 20A are side views of an alternate catheter holder engaged with the annulotomy in accordance with the present invention.

FIGS. 19B and 20B are side views of an alternate catheter holder engaged with the annulotomy in accordance with the present invention.

FIG. 20C is an enlarged side view of an expandable catheter holder positioned to straddle the annulotomy in accordance with the present invention.

FIGS. 21 is a cross-sectional view of an intervertebral disc with an alternate catheter holder in accordance with the present invention.

FIG. 22 is a cross-sectional view of an intervertebral disc with an alternate catheter holder in accordance with the present invention.

FIG. 23 is a side view of the strap of FIG. 22.

FIG. 24 is a cross-sectional view of an intervertebral disc with an alternate catheter holder in accordance with the present invention.

FIG. 25 is a top view of the surgical tool of FIG. 24.

FIG. 26 is a side view of the surgical tool of FIG. 24.

FIG. 27 is a front view of the catheter holder of FIG. 24.

FIG. 28 is a side view of the catheter holder of FIG. 24.

FIG. 29 is a top view of an alternate surgical tool in accordance with the present invention.

FIG. 30 is a side view of the surgical tool of FIG. 29.

FIGS. 31-33 illustrate various views of an alternate mounting flange in accordance with the present invention.

FIG. 34 is a side view of a surgical tool insertion tool in accordance with the present invention.

FIG. 35 illustrates a cross-sectional view of an intervertebral disc of an alternate catheter holder in accordance with the present invention.

FIG. 36 is a side sectional view of an alternate catheter holder in accordance with the present invention.

FIG. 37 is a cross-sectional view of the catheter holder of FIG. 36 engaged with an annulus.

### DETAILED DESCRIPTION OF THE INVENTION

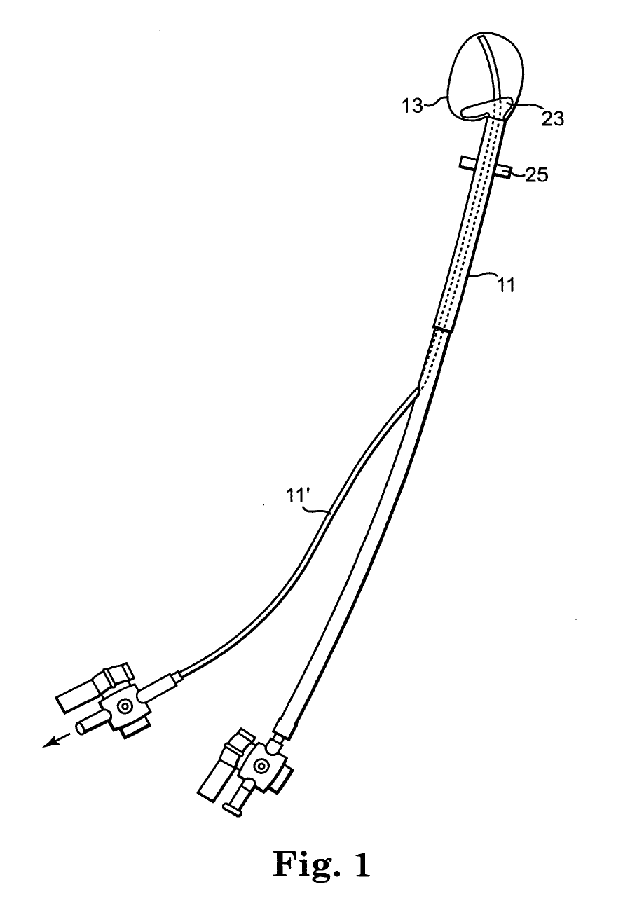

FIG. 1 illustrates an exemplary catheter 11 with mold or balloon 13 located on the distal end for use with the catheter holders of the present invention. In the illustrated embodiment, biomaterial 23 is delivered to the mold 13 through the catheter 11. Secondary tube 11′ evacuates air from the mold 13 before, during and/or after the biomaterial 23 is delivered. The secondary tube 11′ can either be inside or outside the catheter 11, or can enter the mold 13 from another side, such as in a multi-lumen mold. Various multi-lumen molds are disclosed in commonly assigned U.S. patent application Ser. No. ______, entitled Multi-Lumen Mold For Intervertebral Prosthesis And Method Of Using Same filed on the same date herewith (Attorney Docket No. 321297), which is hereby incorporated by reference. Alternatively, the catheter holder of the present invention can be used to inject biomaterial directly into the annulus of a patient, without the use of the mold or balloon 13.

Surgical tool 25 is located on the catheter 11. In the illustrated embodiment, the surgical tool 25 is a stop that limits the depth of insertion of the mold 13 into the annulus (see e.g., FIG. 2). The location of the stop 25 on the catheter 11 can either be fixed or adjustable. In one embodiment, the stop 25 includes a slip-fit mechanism to permit it to be moved to different locations along the catheter 11.

FIGS. 2-7 illustrate a first embodiment of a catheter holder 30 in accordance with the present invention. In the illustrated embodiment, the catheter holder 30 includes a member 32 with a mounting flange 34 on a proximal end 36 and a surgical tool 38 on a distal end 40. The member 32 is preferably rigid or semi-rigid. The member 32 includes a catheter channel 42 adapted to receive catheter 44. In an alternate embodiment, the mounting flange can be attached to the member 32 at a variety of locations. The member 32 can be constructed from a variety of radiopaque or radiolucent materials, such as metal, plastic or a variety of composites. In one embodiment, the member 32 is constructed from a radio-translucent plastic.

In the illustrated embodiment, the mounting flange 34 is attached to a secondary holding device 46 that is preferably attached, directly or indirectly through additional components, to some fixed structure, such as an operating table. In another embodiment, the secondary holding device 46 can include a handle that is gripped by a member of the operating staff to hold the catheter holder 30 in the desired location. In yet another alternate embodiment, the secondary holding device 46 is attached, directly or indirectly through additional components, to the patient, such as for example, using a retractor, Steinmann pins, a harness fitted to the patient, or a variety of other devices.

As used herein, “secondary holding device” refers to a mechanism that can be, directly or indirectly through additional components, releasably attached to the patient, releasably attached to an external structure, gripped by the surgical staff, or any combination thereof. The secondary holding device 46 preferably that limits movement of the catheter holder 30 along at least the z-axis 68, and more preferably along the x-axis, y-axis and z-axis. Although FIG. 2 illustrates the secondary holding device 46 attached near the proximal end 36, the secondary holding device 46 can attach to the catheter holder 30 anywhere along the exposed or accessible portion of the member 32.

The proximal end 36 of the member 32 includes an opening 33 that communicates with the catheter channel 42. The proximal end 36 can be used as an access port for performing other steps in the procedure. For example, the proximal end 36 can be used as a guide for performing the annulotomy 91 (aperture in the annulus 86); performing the nuclectomy (removal of nucleus material 98); evaluating the nuclectomy or the annulus 86; imaging the annulus 86; implanting the mold 13; delivering the biomaterial; and/or cutting the catheter 44 as close to the neck of the mold 13 as possible. Disclosure related to evaluating the nuclectomy or the annulus is found in U.S. patent application Ser. No. 10/984,493, entitled “Multi-Sage Biomaterial Injection System for Spinal Implants, which is incorporated by reference.

The present invention is suitable for accessing the annulus 86 from any of the available access directions, including posterior, posterior lateral, lateral, anterior, or anterolateral. For procedures using the anterior approach, the distal end 40 is preferably longer than used for the posterior approaches.

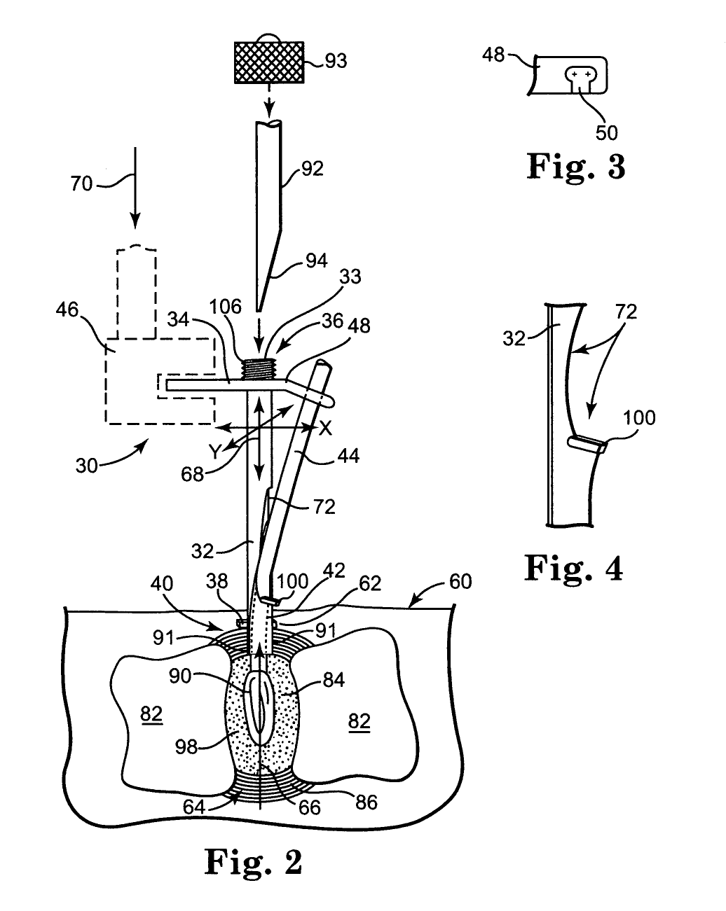

In the illustrated embodiment, the mounting flange 34 also includes extension 48 designed to releaseably engage with the catheter 44. As best illustrated in FIG. 3, the extension 48 in the present embodiment includes a slot 50 which is sized to loosely guide or to compressively engage with the catheter 44. In one embodiment, the slot 50 forms a friction fit with the catheter 44.

In the illustrated embodiment, the surgical tool 38 has a cross-sectional area greater than the cross-sectional area of the distal end 40 of the member 32. The surgical tool 38 of FIG. 2 is adapted to limit the depth of insertion of the member 32 into the annulus 86. In the illustrated embodiment, the surgical tool 38 positions the member 32 against the annulus 86. The surgical tool 38 can optionally serve as a nerve guard ring, a blood vessel retractor, and/or a tamp or support structure for the annulus 86 during injection of the biomaterial, as will be discussed in greater detail below.

As used herein, “surgical tool” refers to one or more of a stop, a tamp or support structure, a blood vessel retractor, and/or a nerve guard ring that attaches near distal end of the member of the present catheter holder. The stop limits the depth of insertion of the catheter into the annulus. The tamp or support structure typically supports an annulus during and/or after delivery of biomaterial. The blood vessel retractor is typically used for anterior or anterolateral entry. The nerve guard ring is typically used for posterior or posterolateral entry. The surgical tools are preferably interchangeable, permitting the surgeon to select one or more while performing the procedure. Various embodiments of the surgical tool are disclosed herein.

As best illustrated in FIGS. 6 and 7, the nerve guard ring 52 includes a support surface 54 with an opening 56 along a portion of its perimeter 58. The nerve guard ring 52 preferably can rotate around the distal end 40 of the member 32, thereby eliminating the need for right-hand and left-hand versions of the present catheter holder 30.

The position of the nerve guard ring 52 along the distal end 40 of the member 32 is preferably adjustable. In one embodiment, the nerve guard ring 52 can move a distance “d” to adjust how far the distal end 40 penetrates into the annulus 86. By adjusting the position of the nerve guard ring 52, the surgeon can precisely control how far bottom edge 41 of the member 32 penetrates into the annulus 86. The bottom edge 41 then can be used as a reference surface or datum for positioning the catheter 44 relative to the catheter holder 30, and in particular, for positioning the optional mold 90 in the cavity 84.

In the illustrated embodiment, the bottom edge 41 of the member 32 has a contour corresponding to the desired shape of the implant. As the mold 90 is filed with biomaterial, it expands against the bottom edge 41, causing it and biomaterial 23 to conform to the shape of the bottom edge 41. When the biomaterial is cured, it will retain the shape of the bottom edge 41. Alternatively, the bottom edge 41 can be flat or a variety of other shapes.

Turning back to FIG. 2, an annulotomy or opening 91 is formed in the annulus 86 of intervertebral disc 64 located between opposing vertebrate 82. The surgical tool 38 limits how far the member 32 penetrates into the patient 60, and in particular, into the annulus 86 and the cavity 84 formed by the nuclectomy. The intervertebral disc 64 and/or tissue 62 adjacent to the intervertebral disc 64 provides a force 66 acting on the surgical tool 38 that limits movement of the catheter holder 30 along the z-axis 68. The tissue surrounding the distal end 40 assists in limiting movement of the distal end 40 along the x-axis and y-axis that intersect the distal end 40.

The secondary holding device 46 limits movement of the proximal end 36 of the catheter holder 30 along the z-axis 68 and preferably along the x-axis and y-axis that intersects the proximal end 36. In one embodiment, the secondary holding device 46 provides a counter acting force 70 generally along the z-axis. In the illustrated embodiment, the forces 66 and 70 assist in retaining the catheter holder 30 relative to the patient 60.

In one embodiment, the member 32 is preferably sufficiently rigid so as to not bend or buckle when subjected to the forces 66 and 70. In another embodiment, the member 32 bends elastically a small amount when subjected to the forces 66 and 70 to applying more constant pressure on the patient 60. In the illustrated embodiment, the forces 66 and 70 are parallel and opposing. In another embodiment, the forces 66 are 70 are co-axial. The forces 66, 70 do not need to be co-axial or parallel to secure the catheter holder 30 to the patient 60. For example, the forces 66, 70 can be at an acute angle relative to each other.

In the illustrated embodiment, member 32 is a hollow tube with a cut-out 72 near the distal end 40. The cut-out 72 is preferably located on the same side of the member 32 as the extension 48. The catheter 44 is inserted into cut-out 72, preferably at an acute angle, through the catheter channel 42 and into the intervertebral disc 64 of the patient 60. Since the position of the catheter holder 30 is fixed relative to the patient 60, the depth to which the catheter 44 penetrates into cavity 84 formed in the annulus 86 can be set and fixed by securing the catheter 44 to the catheter holder 30.

In one embodiment, the catheter 32 is secured to the slot 50 of the extension 48. In another embodiment, catheter locking pin or core 92 is inserted along the z-axis 68 into the proximal end 36 of the catheter holder 30. The tapered tip 94 of the core 92 compresses the catheter 44 against edge 100 in the cut-out 72. In one embodiment, the tapered tip 94 may include a cutting edge. The core 92 can be fixedly engaged with the catheter holder 30 using a cap 93 that has threads that mate with threads 106 on the proximal end 36. The cap 93 can be used to set the proper locking tension on the catheter 44.

Once the catheter 44 is maneuvered along catheter channel 42 to the proper depth within the intervertebral disc 64, biomaterial is delivered through the catheter 44 to mold 90 located in the cavity 84 or directly into the cavity 84 formed in the annulus 86. Various implant procedures, implant molds, and biomaterials related to intervertebral disc replacement suitable for use with the present invention are disclosed in U.S. Pat. No. 5,556,429 (Felt); U.S. Pat. No. 6,306,177 (Felt, et al.); U.S. Pat. No. 6,248,131 (Felt, et al.); U.S. Pat. No. 5,795,353 (Felt); U.S. Pat. No. 6,079,868 (Rydell); U.S. Pat. No. 6,443,988 (Felt, et al.); U.S. Pat. No. 6,140,452 (Felt, et al.); U.S. Pat. No. 5,888,220 (Felt, et al.); U.S. Pat. No. 6,224,630 (Bao, et al.), and U.S. patent application Ser. Nos. 10/365,868 and 10/365,842, all of which are hereby incorporated by reference.

In some embodiments, the annulus 86 can itself serve as a suitable mold for receiving the biomaterial. Optionally, the interior surface of the annular shell can be treated or covered with a suitable material in order to enhance its integrity and use as a mold. Although the embodiments herein disclose the use of a mold mounted on the end of the catheter, the present catheter holders are equally applicable to the delivery of biomaterial directly into the annulus.

The catheter holder and method of the present invention can also be used to repair other joints, including diarthroidal and amphiarthroidal joints. Examples of suitable diarthroidal joints include the ginglymus (a hinge joint, as in the interphalangeal joints and the joint between the humerus and the ulna); throchoides (a pivot joint, as in superior radio-ulnar articulation and atlanto-axial joint); condyloid (ovoid head with elliptical cavity, as in the wrist joint); reciprocal reception (saddle joint formed of convex and concave surfaces, as in the carpo-metacarpal joint of the thumb); enarthrosis (ball and socket joint, as in the hip and shoulder joints) and arthrodia (gliding joint, as in the carpal and tarsal articulations). The present catheter holders can also be used for a variety of other procedures, including those listed above.

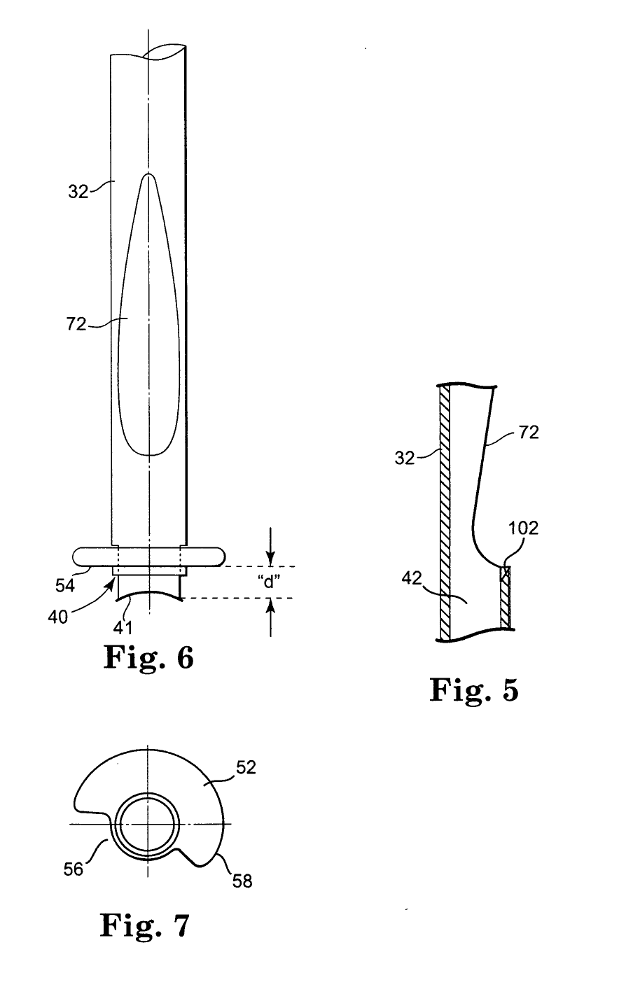

As best illustrated in FIG. 4, the cut-out 72 in the member 32 optionally includes a flared edge 100 designed to minimize damage to the catheter 44. FIG. 5 illustrates an alternate embodiment of the member 32 with an angled groove 102 located at the lower end of the cut-out 72. The groove 102 preferably has a diameter slightly larger than the diameter of the catheter 44. The cut-out 72 is preferably configured to create a smooth transition of the catheter 44 into the catheter channel 42.

The core 92 can optionally be used to cut the catheter 44 after the biomaterial is cured. In one embodiment, the core 92 is rotated or advanced further into the member 32, severing catheter 44 and cured biomaterial at a location near the edge 100. For example, the threads 106 can be used to advance the core 92 toward the edge 100. Alternatively, the core 92 is removed and the catheter 44 is manually cut near the edge 100.

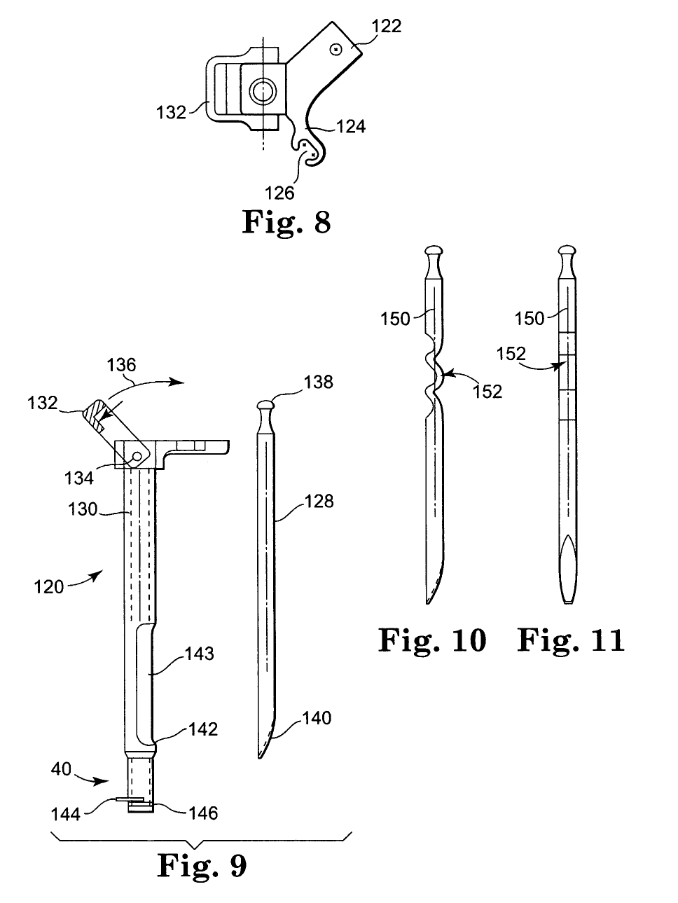

FIGS. 8 and 9 illustrate an alternate catheter holder 120 in accordance with the present invention. As best illustrated in FIG. 8, mounting flange 122 is offset from extension 124 by approximately 90 degrees. Extension 124 includes a slot 126 sized to receive a catheter. The catheter holder 120 includes a core 128 which is positioned in the center of the member 130, as discussed above. Once the core 128 is positioned in the member 130, bail 132 is rotated around pivot point 134 along the direction 136 to secure proximal end 138 of the core 128 within the member 130. The tapered end 140 of the core 128 secures a catheter against the edge 142 of the cut-out 143, as discussed above.

Distal end 40 of the member 130 includes surgical tool 144 as discussed above. An optional radiopaque marker band 146 can be located on the distal end 40 of the member 130. The radiopaque band 146 can optionally be used for imaging during the implant procedure.

FIGS. 10 and 11 illustrate front and side views of an alternate core 150 for use in the member 130 of FIG. 9. The core 150 includes a spring region 152 that assists in applying more constant pressure on the catheter. In the illustrated embodiment, the spring region 152 is a serpentine or corrugated region formed in the core 150. In an alternate embodiment, the core 150 can be a two-piece telescoping structure with an internal spring to provide a predictable spring force. The spring region 152 is particularly useful in accommodating for tolerances and materials when working with plastic parts.

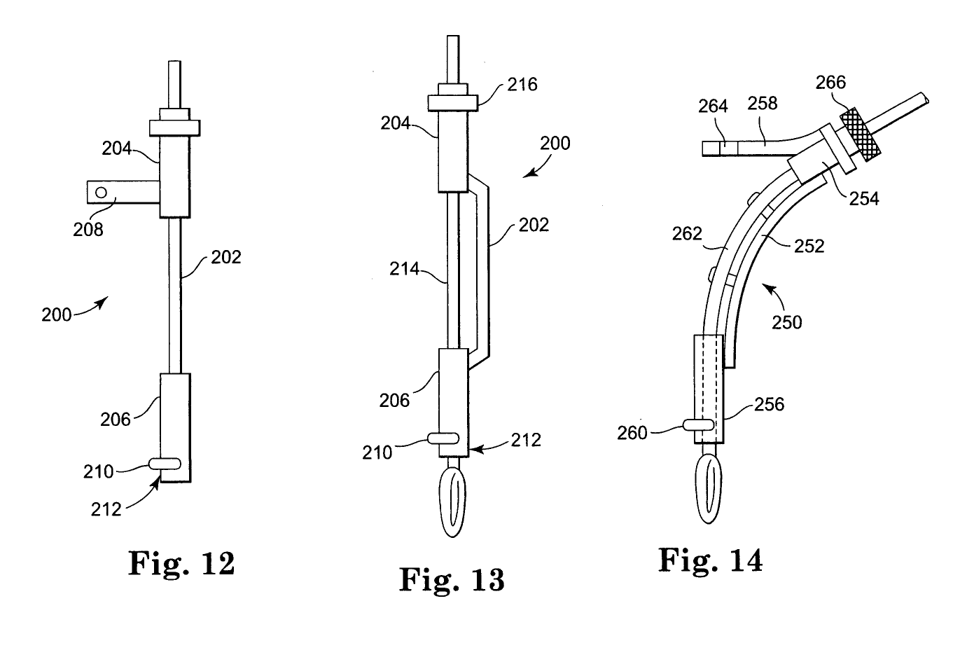

FIGS. 12 and 13 illustrate an alternate catheter holder 200 in accordance with the present invention. The catheter holder 200 includes a member 202 with an upper tubular member 204 and a lower tubular member 206. Mounting flange 208 is preferably attached to the upper tubular member 204 or the member 202. Surgical tool 210 is attached at the distal end 212 of the lower tubular member 206. Catheter 214 is inserted through the tubular members 204 and 206 as discussed above. Tubular member 204 preferably includes a compression member 216 to secure the catheter 214 relative to the length of the catheter holder 200.

FIG. 14 illustrates an alternate catheter holder 250 in accordance with the present invention. The catheter holder 250 includes a curved member 252 with an upper tubular member 254 and a lower tubular member 256. Mounting flange 258 is attached to the upper tubular member 254. The mounting flange 258 preferably includes an opening 264 co-axially aligned with lower tubular member 256. The opening 264 and the lower tubular member 256 are provided to receive and position other devices used in the implant procedure, such as imaging catheters, catheter cutters, and the like.

Surgical tool 260 is attached to the lower tubular member 256. Catheter 262 is inserted through the tubular members 254, 256 and into the patient as discussed above. Compression member 266 is preferably provided on the upper tubular member 254 to secure the catheter 262 relative to the length of the catheter holder 250.

FIG. 15 is a side view of an alternate catheter holder 300 in accordance with the present invention. Member 302 includes a tubular portion 304 on the proximal end 308 and tubular portion 306 on the distal end 310. The tubular portions 304, 306 are provided to receive and position other devices used in the implant procedure, such as imaging catheters, catheter cutters, and the like.

Mounting flange 312 is attached to the tubular member 304 on the proximal end. Surgical tool 314 is attached to the tubular member 306 on the distal end 310. Catheter guide 316 is positioned at an angle relative to the member 302. Extension 318 attaches the distal end 306 of the member 302 to the catheter guide 316. Compression screw 320 is provided on the catheter guide 316 to secure a catheter relative to the catheter holder 300.

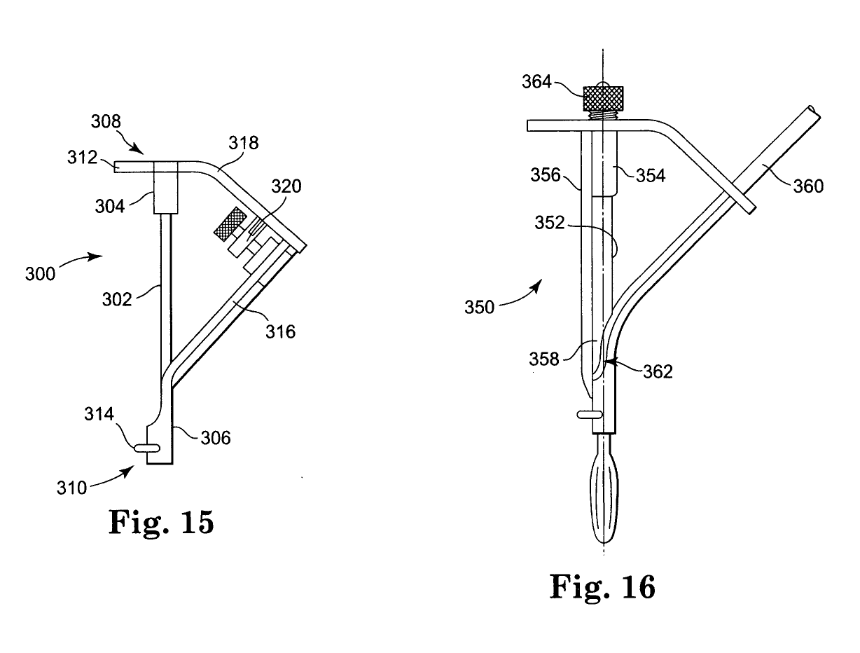

FIG. 16 is a side view of an alternate catheter holder 350 in accordance with the present invention. Catheter locking pin 352 is positioned through the tubular portion 354 of the member 356. Distal end 358 of the catheter locking pin 352 compressively engages with catheter 360 against edge 362 of the member 356 to secure it in place relative to the catheter holder 350. Threaded member 364 can be used to advance the catheter locking pin 352 toward the catheter 360, such as for example to fix the position of the catheter 360 relative to the catheter holder 350, or to cut the catheter 360 and cured biomaterial contained within using a sharpened distal end 358.

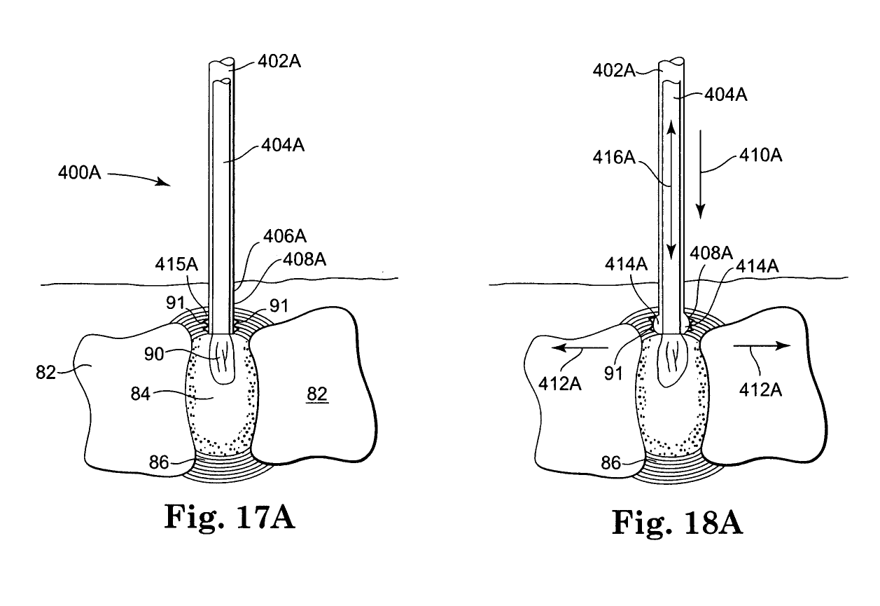

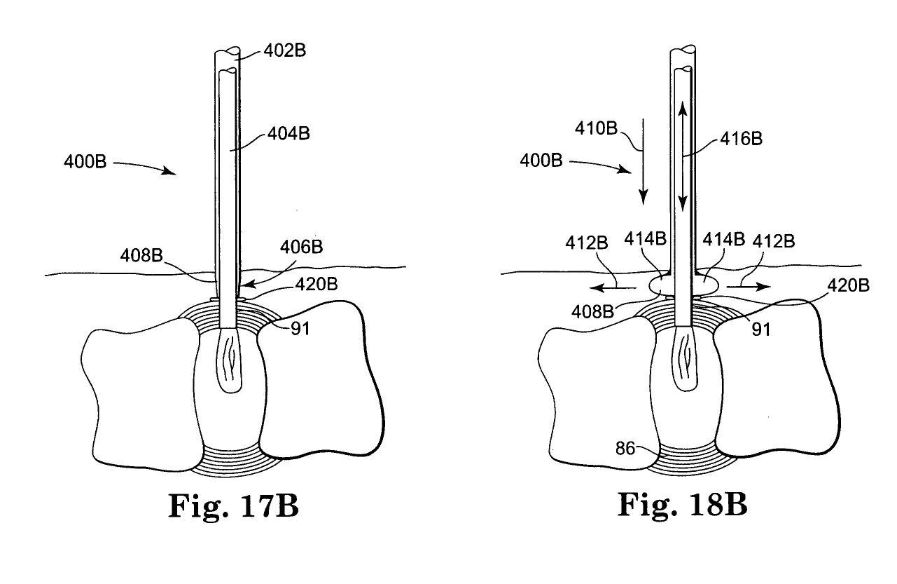

FIGS. 17A and 18A illustrate an alternate catheter holder 400A that can optionally be used without a secondary holding device. The catheter 404A is secured to the annulus 86, and in particular, within the annulotomy 91.

Outer sleeve 402A surrounds the catheter 404A. Distal end 406A of the outer sleeve 402A is attached to the catheter 404A by a flexible material 408A. The flexible material 408A can be rubber, silicone, or any other elastically or plastically expandable material. In one embodiment, the flexible material 408A forms a continuous structure, such as for example a disc or a toroid. In another embodiment, the flexible material 408A can be a multi-lobed structure, which can optionally be constructed from metal.

As best illustrated in FIG. 18A, the outer tube 402A is displaced in the direction 410A relative to the catheter 404A, causing the flexible material 408A to deform in an outward direction 412A. Protrusions 414A expand into the edges of the annulotomy 91 in the annulus 86, forming an interference or compression fit. The present embodiment secures the catheter holder 400A from movement in either direction along the axis 416A, preferably without a secondary holding device.

In one embodiment, the external surface of the protrusions 414A can optionally include a variety of surface features 415A to releasably engage with the edge of the annulotomy 91, such as for example ridges, spikes, and the like. The surface features serve to more securely anchor the protrusions 414A to the annulotomy 91. A variety of the embodiments disclosed herein can also benefit from the use of such surface features. In one embodiment, the resulting protrusions 414A operate as a surgical tools such as a stop, discussed in other embodiments herein.

FIGS. 17B and 18B illustrate an alternate catheter holder 400B in which the protrusions 414B are deployed subcutaneous, outside of the annulus 86. In this embodiment, the surgical tool 420B is positioned against the edge of the annulotomy 91 establishes a frame of reference for the procedure, providing greater accuracy. The outer tube 402B is displaced in the direction 410B relative to the catheter 404B as discussed above, causing the flexible material 408B to deform in an outward direction 412B. The present embodiment secures the catheter holder 400B from movement in either direction along the axis 416B, preferably without a secondary holding device. In one embodiment, the resulting protrusions 414B operate as a surgical tools making the stop 420B unnecessary, discussed in other embodiments herein.

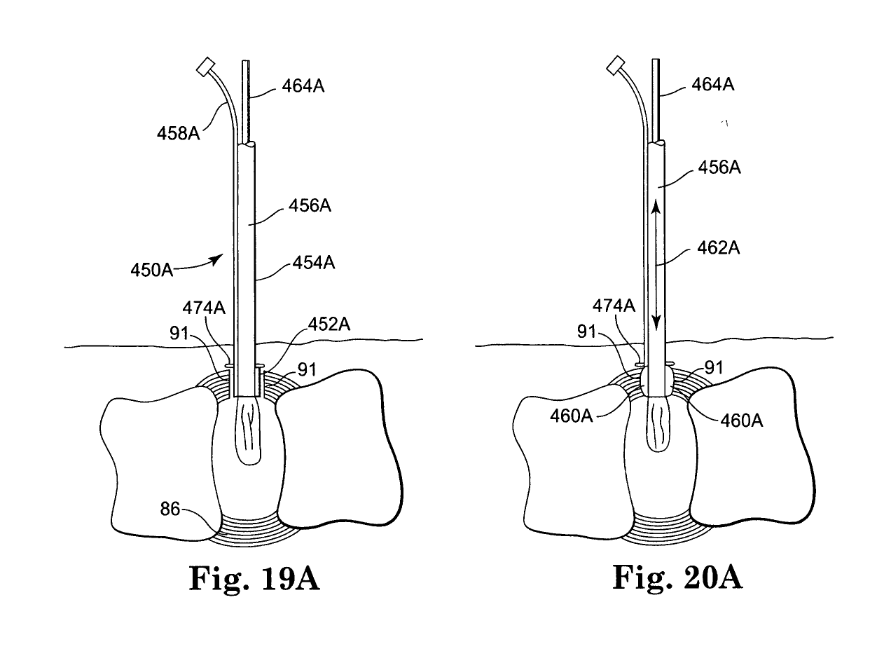

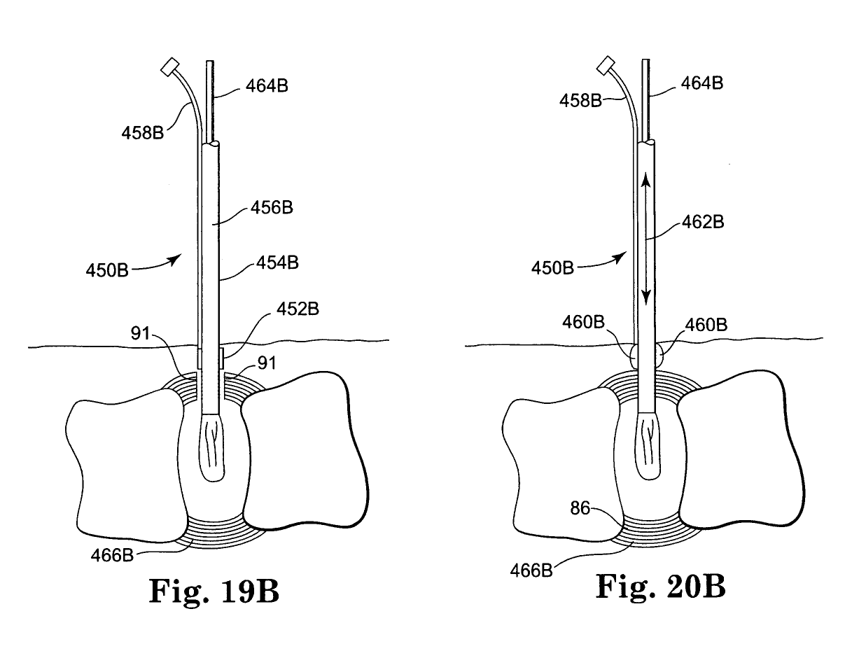

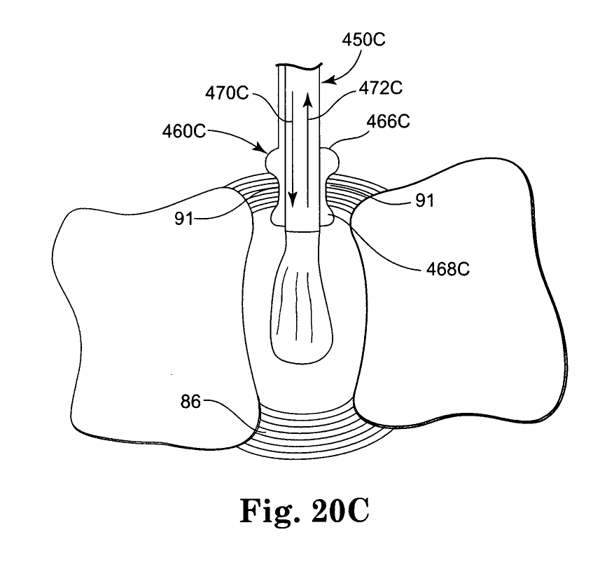

FIGS. 19A and 20A illustrate an alternate catheter holder 450A that can optionally be used without a secondary holding device. The catheter 464A is secured to the annulus 86, and in particular, within the edges of the annulotomy 91. Expandable bladder 452A is fitted on external surface 454A of tubular member 456A. Delivery tube 458A is in fluid communication with the expandable bladder 452A.

The expandable bladder 452A is positioned within the annulotomy 91. As best illustrated in FIG. 20A a fluid is delivered to the expandable bladder 452A through the delivery tube 458A, causing it to inflate. The inflated bladder 460A forms an interference or compression fit within the annulotomy 91. The inflated bladder 460A serves to secure the tubular member 456A along either direction of axis 462A, using the edge of the annulotomy 91 and the surgical tool 474A as the frame of reference for the procedure. Delivery catheter 464A, or a variety of other devices, can be inserted through the tubular member 456A. In an alternate embodiment, the bladder 452A can be located directly on the catheter 464A, obviating the tubular member 456A. In one embodiment, the inflated bladder 460A operates as a surgical tool such as a stop, making the surgical tool 474A unnecessary.

FIGS. 19B and 20B illustrate an alternate catheter holder 450B in accordance with the present invention. The expandable bladder 452B is positioned outside the annulotomy 91. As best illustrated in FIG. 20B a fluid is delivered to the expandable bladder 452B through the delivery tube 458B, causing it to inflate. The inflated bladder 460B is deployed subcutaneous, outside of the annulus 86. The inflated bladder 460B forms an interference or compression fit within the tissue outside of the annulus 86 to secure the tubular member 456B along either direction of axis 462B. The inflated bladder 460B can alternately be located in the annulotomy 91, outside the annulus 86, inside the annulus 86, or a combination thereof. In one embodiment, the inflated bladder 460B operates as a surgical tools such as a stop.

FIG. 20C illustrates an alternate interface of the inflated bladder 460C with the annulus 86. The inflated bladder 460C extends across the depth of the annulotomy 91 so that a portion 466C is located outside the annulus 86 and a portion 468C is located inside the annulus 86. In the embodiment of FIG. 20C, the inflated bladder 460C has a generally hourglass shape. The portion 466C limits movement of the catheter holder 450C in the direction 470C and the portion 468C limits movement of the catheter holder 450C in the direction 472C.

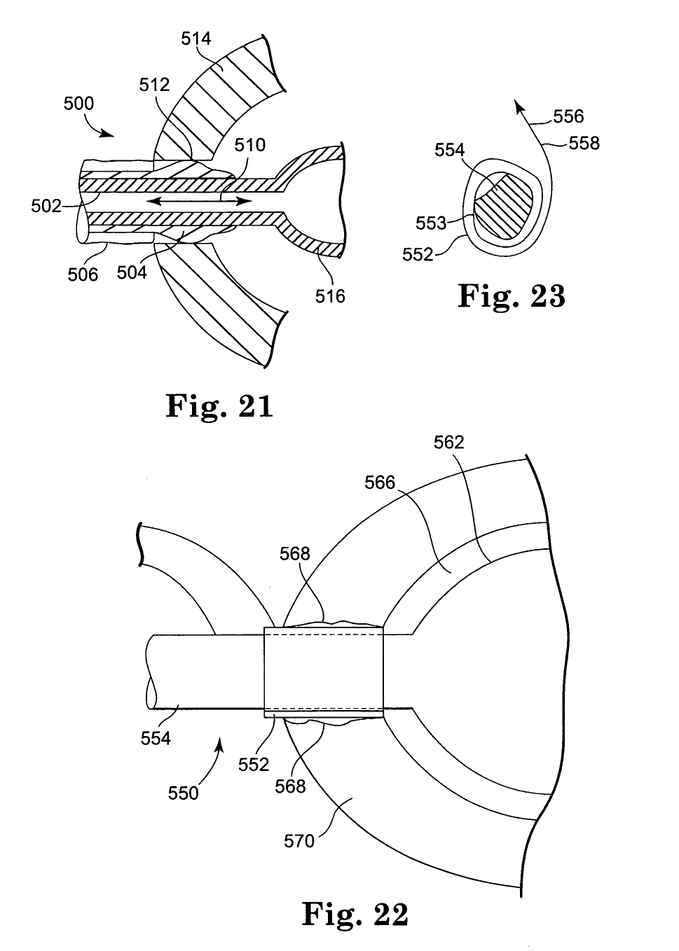

FIG. 21 is a side sectional view of an alternate catheter holder 500 in accordance with the present invention. Catheter 502 is optionally fitted with an expandable bladder 504. Sheath 506 can optionally be slid over the delivery tube 502 and expandable bladder 504 to control the location and degree of expansion of the bladder 504. In the illustrated embodiment, the expanded bladder 504 engaged with edges of the annulotomy 512 in the annulus 514. The embodiment of FIG. 21 is particularly useful when biomaterials are injected into the annulus 512 without mold 516, since the bladder 504 not only fixes the catheter holder 500 relative to the annulus 86, it also seals the annulotomy 91 to allow biomaterial to be injected under pressure without leaking out. As discussed above, the inflated bladder 504 limits movement of the catheter holder 500 along either direction of the axis 510, making it unnecessary to secure the proximal end of the catheter 502 to the surgical table or other fixed structure.

FIGS. 22 and 23 illustrate an alternate catheter holder 550 in accordance with the present invention. Expandable strap 552 is positioned around the catheter 554. Proximal end 553 of the expandable strap 552 is preferably attached to the catheter 554.

As best illustrated in FIG. 23, when force 556 is applied to distal end 558 of this strap 552, the portion surrounding the catheter 554 contracts concentrically inward. The mold 562 attached to the distal end of the catheter 554 is then positioned in the cavity 566 formed inside annulus 570. When the force 556 is released, the strap 552 expands to its substantially original shape to form a friction or interference fit with the annulotomy 568 in the annulus 570. When the procedure is completed, the surgeon reapplies the force 556 to contract the strap 552, and the strap 552 is removed from the annulotomy.

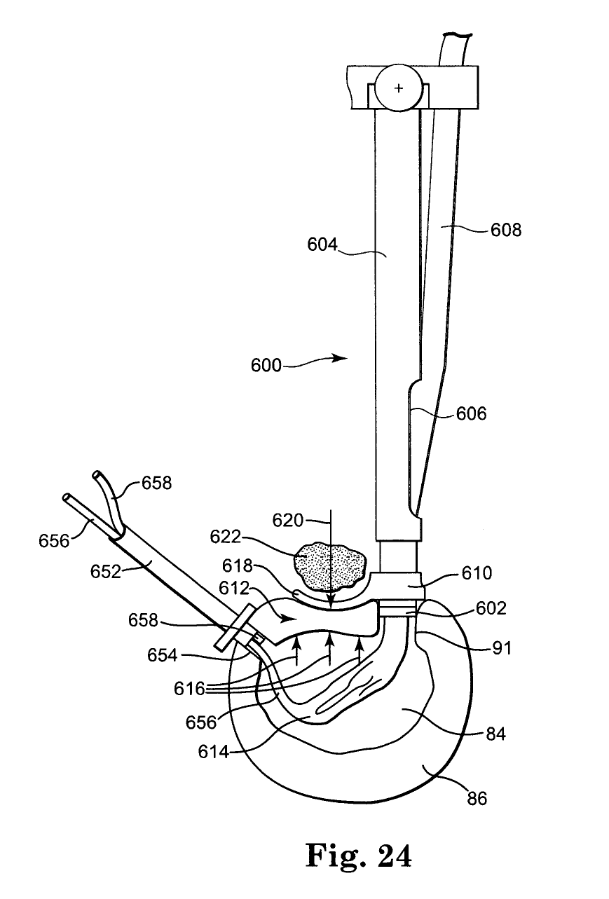

FIG. 24 is a cross-sectional view of an annulus 86 engaged with an alternate catheter holder 600 in accordance with the present invention. Distal end 602 of member 604 includes cut-out 606 that permits catheter 608 to be inserted into the annular cavity 84 formed in the annulus 86. In the illustrated embodiment, surgical tool 610 operates to support region 612 of the annulus 86 and to retract and protect blood vessels and nerves. In the illustrated embodiment, the surgical tool 610 can also operate as a nerve guard ring.

In operation, as a fluid, such as a curable biomaterial, is delivered through the catheter 608, the mold 614 inflates within the cavity 84. Alternatively, the biomaterial is injected directly into the annulus 86 without use of the mold 614. In some applications, the inflated mold 614 or the biomaterial located in the annulus 86 exert pressure 616 on the annulus 86, such as region 612. Portion 618 of the surgical tool 610 provides a counteracting force 620 that restrains deformation of the annulus 86. In particular, the portion 618 prevents the annulus 86 from impinging on spinal column 622. The surgical tool 610 is particularly useful when the portion 612 of the annulus 86 is diseased or otherwise weakened, and hence, prone to distend or deform when subjected to the pressure of the biomaterial.

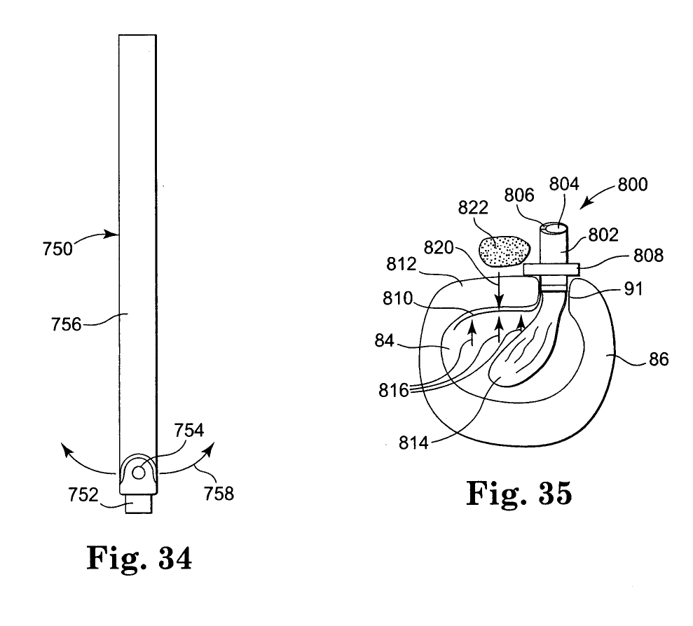

Depending upon the size of the portion 618, the surgical tool 610 can optionally be positioned in the patient before the member 604. FIG. 34 illustrates one possible embodiment of an insertion tool 750 in accordance with the present invention. Member 754 forms an articulating connection between distal end 752 and the member 756. Member 754 articulates through at least one degree of freedom, and preferably two or more degrees of freedom. In the illustrated embodiment, member 754 is a pivot point around which the distal end 752 rotates along arc 758 relative to the member 754. The distal end 752 mechanically engages with a surgical tool, such as the surgical tools in FIGS. 26 and 30.

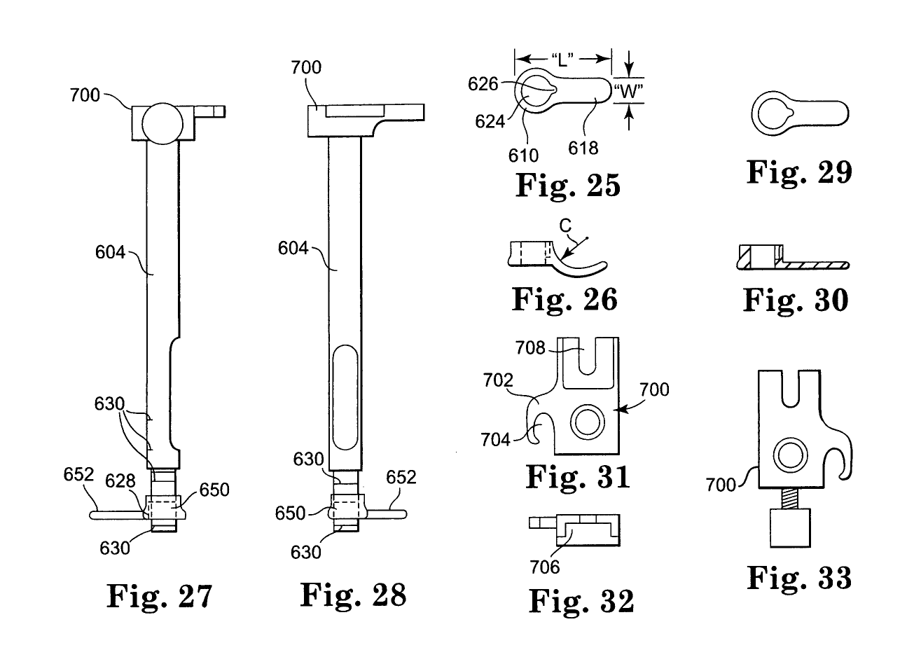

In the embodiment of FIG. 24 the surgical tool 610 is preferably releasably attached to the distal end 602 of the member 604, such as by a snap-fit arrangement, a mechanical release or a variety of other mechanisms. In particular, the surgeon inserts the surgical tool 610 into the patient against the annulus 86. The opening 624 (see FIGS. 25-26) on the surgical tool 610 is aligned with the annulotomy 91. The catheter holder 600 is then inserted into the annulotomy 91 so that the distal end 602 engaged with the opening 624 on the surgical tool 610. Once the mold 614 or the annulus 86 is filled with curable biomaterial, the sequence is reversed to remove the surgical tool 610 from the patient.

In an alternate embodiment, second catheter holder 652 is optionally engaged with posterolateral annulotomy 654, in accordance with the present invention. In the illustrated embodiment, the catheter holder 652 includes a second lumen 656 fluidly coupled to the mold 614 and a visualization device 658, such as an endoscope.

The second catheter holder 652 provides a second discrete access port 654 into the annulus 86 that optionally can be used to form the annular cavity 84, to image any phase of the procedure, to deliver the biomaterial to the mold 614 through the second lumen 656, to draw a vacuum on the mold 614 before, during and/or after delivery of the biomaterial, and to secure the prosthesis in the intervertebral disc space during and after delivery of the biomaterial. Various multi-lumen molds and mechanisms for securing a prosthesis in the annulus are disclosed in U.S. Provisional application entitled Multi-Lumen Mold for Intervertebral Prosthesis and Method of Using Same, filed on the same date herewith (attorney docket no. 319570), which is hereby incorporated by reference.

As best illustrated in FIGS. 25 and 26, the surgical tool 610 includes an opening 624 sized to engage with distal end 602 of the member 604. An optional anti-rotation feature 626, such as a notch, is provided to engage with a corresponding feature 628 on the distal end 602. The portion 618 includes a width “W”, a length “L” and an optional curvature “C”. The desired width, length and curvature can vary with the patient. In another embodiment, the portion 618 has a curvilinear shape with varying radii of curvature. In another embodiment, the portion 618 has a constant radius of curvature. The present invention includes a kit having a plurality of interchangeable surgical tools 610 having a variety of widths, lengths, and curvatures.

FIGS. 27 and 28 illustrate front and side views of the member 604 of FIG. 24 with an alternate surgical tool 650 engaged with distal end 602. The member 604 is preferably constructed from a radio-translucent plastic, with a plurality of radiopaque markers 630 attached or embedded therein. Alternatively, the member 604 is constructed from a radiopaque material. The radiopaque markers 630 can have a variety of shapes and can be arranged in a variety of configurations, such as for example the straight lines arranged at fixed intervals illustrated in FIGS. 27 and 28.

As best illustrated in FIGS. 29 and 30, the surgical tool 650 includes the same opening 624 and anti-rotation feature 626 as the surgical tool 610. The surgical tools 610 and 650 are preferably interchangeable with the distal end 602 of the member 604.

FIGS. 31-33 illustrate an alternate mounting flange 700 in accordance with the present invention. Extension 702 includes a slot 704 sized to receive a catheter 608. Recess 706 and slot 708 are preferably configured to engage with a mounting arm attached to a fixed location, such as the surgical table. Once such mounting arm is a component of a micro-discectomy system sold by Medtronic under the trade name MetrX®.

FIG. 35 is a side view of an alternate catheter holder 800 in accordance with the present invention. Member 802 includes a primary lumen 804 and a secondary lumen 806. In the illustrated embodiment, 804 is a working lumen and 806 is a visualization lumen. The relative sizes of the lumens 804, 806 are schematically illustrated and can vary with the application.

Surgical tool 808 engages with the annulus 86 adjacent to the annulotomy 91. The location of the surgical tool 808 along the member 802 is optionally adjustable with a snap-fit sliding motion. A second surgical tool, reinforcing member 810 is delivered to the cavity 84 into the annulus 86 preferably through the member 802. The reinforcing member 810 operates to support region 812 of the annulus 86. The reinforcing member 810 is optionally a shaped memory metal, such as for example nitinol.

In operation, as a fluid, such as a curable biomaterial, is delivered through the catheter 804, the mold 814 inflates within the cavity 84. In some applications, the inflated mold 814 exerts pressure 816 on the annulus 86, such as at the region 812. Alternatively, in an embodiment where the biomaterial is injected directly into the annulus 86 without the use of a mold 814, the biomaterial exerts pressure 816 on the region 812.

The surgical tool 810 provides a counteracting force 820 and/or distributes the force 816 over a larger surface area to temporarily reinforce the region 812 of the annulus 86. In particular, the surgical tool 810 prevents the annulus 86 from protruding into the spinal cord 822. The surgical tool 810 is particularly useful when the portion 812 of the annulus 86 is diseased or otherwise weakened, and hence, prone to distend or deform when subjected to the pressure of the inflated mold 814. Preferably after the biomaterial is at least partially cured, the reinforcing member 810 is removed from the annulus 86 through the member 802. In an alternate embodiment, the surgical tool 810 can engage with an exterior surface of the annulus 86, such as illustrated in FIG. 24.

In another embodiment, the surgical tool 810 is released from the member 802 and remains in the annulus 86 after the procedure is completed. The surgical tool 810 is preferably release from the member 802 after the biomaterial is at least partially cured. In one embodiment, the surgical tool 810 is constructed from a bio-resorbable material.

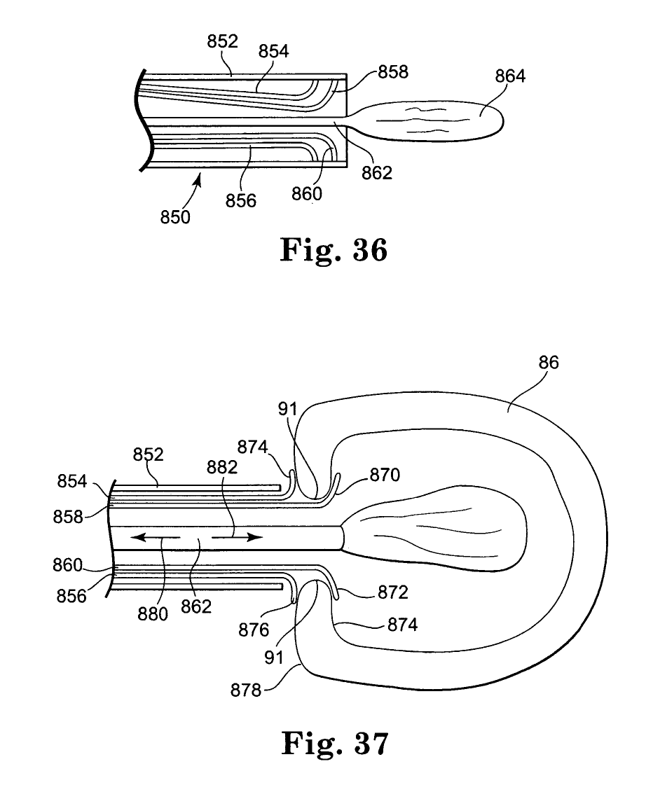

FIG. 36 is a side sectional view of an alternate catheter holder 850 in accordance with the present invention. The member 852 includes one or more outer engagement wires 854, 856 and one or more inner engagement wires 858, 860. Catheter 862 and mold 864 are preferably located in center region 866 of member 852.

As illustrated in FIG. 37, member 852 is located adjacent to annulus 86. The inner engagement wires 858, 860 are extended from the member 852 so that curved portions 870, 872 engage with inner surface 874 of the annulus 86 adjacent to the annulotomy 91. The outer engagement wires 854, 856 are also extended from the member 852 so that curved portions 874, 876 engage with outer surface 878 of the annulus 86 adjacent to the annulotomy. The engagement wires 858, 860 prevent the catheter holder 850 from moving in a direction 880, while the engagement wires 854, 856 prevent movement in direction 882. The catheter holder 850 can be used with or without a secondary holding device. The catheter 862 can be positioned in and secured to the member 852 using any of the methods disclosed herein.

The catheter holder of the present invention can also be used with the method of implanting a prosthetic nucleus disclosed in a commonly assigned U.S. Patent Application entitled Lordosis Creating Nucleus Replacement Method And Apparatus (Attorney Docket No. 318946), filed on the same date herewith, the disclosure of which are incorporated herein by reference.

Patents and patent applications disclosed herein, including those cited in the Background of the Invention, are hereby incorporated by reference. Other embodiments of the invention are possible. Many of the features of the various embodiments can be combined with features from other embodiments. In particular, any of the present catheter holder can be combined with the surgical tools, multi-lumen molds and/or entry ports discussed herein. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A catheter holder attachable to a secondary holding device for the in situ formation of a prosthesis in an intervertebral disc space of a patient using minimally invasive techniques, the catheter holder comprising:
a member having at least one catheter channel, a proximal end and a distal end configured to extend into the intervertebral disc space, the distal end having a distal opening in fluid communication with the catheter channel;
a mounting flange attached to the member and attachable to the secondary holding device;
at least one surgical tool located near the distal end of the member; and
a securing mechanism adapted to secure a catheter extending through the catheter channel to the catheter holder.

2. The catheter holder of claim 1 wherein the proximal end comprises a proximal opening in fluid communication with the catheter channel.

3. The catheter holder of claim 1 comprising an aperture in a side of the member fluidly coupled to the catheter channel configured to direct a catheter into the catheter channel at an acute angle.

4. The catheter holder of claim 1 wherein the securing mechanism comprises a catheter locking pin sized to fit into the catheter channel, the catheter locking pin having a distal end configured to compressively secure a catheter in the catheter channel.

5. The catheter holder of claim 4 wherein the catheter locking pin comprises a spring structure.

6. The catheter holder of claim 1 wherein the securing mechanism comprises a compression fitting adapted to secure a catheter in the catheter channel.

7. The catheter holder of claim 1 comprising a catheter with a mold attached to a distal end thereof, the mold having at least one lumen extending through the catheter holder.

8. The catheter holder of claim 1 wherein the surgical tool comprises a plurality of interchangeable stops each adapted to limit a depth of insertion into the intervertebral disc space.

9. The catheter holder of claim 8 wherein the plurality of interchangeable stops comprise two or more stops having different profiles.

10. The catheter holder of claim 1 wherein the surgical tool comprises one or more of a support structure, a nerve guard ring, a blood vessel retractor or a tamp.

11. The catheter holder of claim 10 wherein the support structure comprising a plurality of interchangeable support structures having two or more different profiles.

12. The catheter holder of claim 10 wherein the support structure comprises a shape adapted to engage with an external surface of an annular body located in the intervertebral disc space.

13. The catheter holder of claim 10 wherein the support structure comprises a shape adapted to extend into an intervertebral disc located in the intervertebral disc space.

14. The catheter holder of claim 10 wherein the support structure is adapted to be deposited in an intervertebral disc.

15. The catheter holder of claim 10 wherein the support structure comprises a bio-resorbable material.

16. The catheter holder of claim 1 comprising at least one retractable surgical tool extendable through the catheter channel into the intervertebral disc space.

17. The catheter holder of claim 16 wherein the retractable surgical tool comprises a shaped memory metal.

18. The catheter holder of claim 1 wherein the surgical tool comprises a plurality of engagement wires located in the member and adapted to project from the distal end of the member to engage with an intervertebral disc.

19. The catheter holder of claim 1 comprising a second catheter holder having a distal end configured to extend into the intervertebral disc space.

20. The catheter holder of claim 19 comprising a visualization device located in the second catheter holder.

21. The catheter holder of claim 1 wherein the member comprises one of a radiopaque material or a radio-translucent material.

22. The catheter holder of claim 1 wherein the catheter channel includes at least one visualization device.

23. The catheter holder of claim 1 comprising:
a catheter located in the catheter channel; and
a flexible material attaching the distal end of the member to the catheter, such that displacement of the member relative to the catheter causes the flexible material to expand outwardly away from the distal end.

24. The catheter holder of claim 1 comprising:
an outer tube surrounding the member; and
a flexible material attaching the distal end of the member to the distal end of the outer tube, such that displacement of the outer tube toward the distal end of the member causes the flexible material to expand outwardly away from the distal end of the member.

25. The catheter holder of claim 1 comprising:
an expandable bladder located near at least the distal end of the member; and
a delivery tube fluidly coupled to the expandable bladder.

26. The catheter holder of claim 25 comprising a sheath extending around a portion of the expandable bladder.

27. The catheter holder of claim 1 comprising an expandable coiled material having a proximal end attached to the tubular member near the distal end, the expandable coiled material having an outer diameter that contracts when a force directed away from the tubular member is applied to a distal end of the expandable coiled material.

28. A catheter holder for the in situ formation of a prosthesis in an intervertebral disc space of a patient using minimally invasive techniques, the catheter holder comprising:
a catheter having a distal end;
an outer tube have a distal end surrounding the catheter; and
a flexible material attaching the distal end of the catheter to the distal end of the outer tube, such that displacement of the outer tube relative to the catheter causes the flexible material to expand outwardly away from the catheter.

29. A method for the in situ formation of a prosthesis in an intervertebral disc space of a patient using minimally invasive techniques, the method comprising the steps of:
positioning a distal end of a member in the intervertebral disc space, the distal end having a distal opening in fluid communication with a catheter channel in the member;
engaging at least one surgical tool located on the distal end of the member with the intervertebral disc space;
securing the member to a secondary holding device;
inserting a catheter through a catheter channel into the intervertebral disc space; and
securing the catheter to the member.

30. The method of claim 29 comprising the step of selecting a surgical tool from a plurality different surgical tools each engageable with the distal end of the member.

31. The method of claim 29 wherein the step of securing the catheter to the member comprises inserting a catheter locking pin into an opening at the proximal end of the member so that distal end of the catheter locking pin compressively secures the catheter in the catheter channel.

32. The method of claim 29 comprising the step of delivering a curable biomaterial through the catheter to the intervertebral disc space.

33. The method of claim 29 comprising the step of delivering a curable biomaterial through the catheter to a mold located in the intervertebral disc space.

34. The method of claim 29 comprising locating radiopaque markers on the member.

35. The method of claim 29 comprising the steps of:
attaching a flexible material to the distal end of the member and to the catheter; and
displacing the member relative to the catheter so that the flexible material expands outwardly away from the distal end.

36. The method of claim 29 comprising the steps of:
positioning an outer tube around the member; and
attaching the distal end of the member to a distal end of the outer tube using a flexible material; and
displacing the outer tube toward the distal end of the member so that the flexible material expands outwardly away from the distal end of the member.

37. The method of claim 29 comprising the steps of:
locating an expandable bladder proximate the distal end of the member; and
delivering a fluid to the bladder so that the expanded bladder forms an interference fit with the patient.

38. The method of claim 37 comprising locating a sheath around the expandable bladder to selectively control the expansion thereof.

39. The method of claim 29 comprising the steps of:
attaching a proximal end of an expandable coiled material to the tubular member near the distal end;
applying a force directed away from the tubular member to contract the coiled material;
positioning the expandable coiled material in the patient; and
releasing the force so that the coiled material expands and forms an interference fit with the patient.

40. The method of claim 29 comprising the steps of:
inserting a surgical tool through the member and into the intervertebral disc space;
delivering a biomaterial to the intervertebral disc space; and
retracting the surgical tool from the intervertebral disc space.
